(19) United States
(12) Patent Application Publication
(10) Pub. No.: US 2005/0003405 A1
(43) Pub. Date: Jan. 6, 2005

(54) Treatment and diagnostics of cancer

(76) Inventors: Hua-Chien Chen, Taipei (TW); Ying Sun, London (GB); Ying-Huey Huang, Changhua (TW); Ming-Chu Hsu, Glendora, CA (US); Din-Lii Lin, Arcadia, CA (US)

Correspondence Address: FISH & RICHARDSON PC, 225 FRANKLIN ST, BOSTON, MA, 02110, US

(21) Appl. No.: 10/833,708

(22) Filed: Apr. 27, 2004

Related U.S. Application Data
(60) Provisional application No. 60/466,599, filed on Apr. 30, 2003.

(51) Int. Cl. C12Q 1/68; G01N 33/574

(52) U.S. Cl. 435/6; 435/7.23

(57) ABSTRACT

A method of determining whether a subject is suffering from or at risk for developing cancer. The method involves providing a sample from a subject, and determining the level of HM74, LGR6, GPR88, or GPR49 gene expression or protein activity in the sample. The level of HM74, LGR6, GPR88, or GPR49 gene expression or protein activity in the sample, if higher than that in a sample from a normal subject, indicates that the subject is suffering from or at risk for developing cancer. Also disclosed are a method of identifying a compound for treating cancer, a method of treating cancer, and a pharmaceutical composition or a packaged product for treating cancer.

### RELATED APPLICATION

This application claims priority to U.S. Provisional Application Ser. No. 60/466,599, filed Apr. 30, 2003, the contents of which are incorporated herein by reference.

### BACKGROUND

G protein-coupled receptors (GPCRs) are the largest and most diverse family of transmembrane receptors. Responding to a wide range of stimuli including small peptides, lipid analogs, amino-acid derivatives, and sensory stimuli such as light, taste, and odor, they transmit signals to the interior of the cell through interaction with heterotrimeric G proteins. It has been estimated that, of the 35,000 or so human genes, approximately 750 are GPCRs. About half of these sequences are likely to encode sensory receptors, leaving nearly 400 receptors that can be considered as potential targets for drug development (Sautel and Milligan (2000) Curr Med Chem 7(9), 889-896; and Gurrath (2001) Curr Med Chem 8(13), 1605-1648). GPCRs are widely expressed and mediate most cell-cell communication in humans. Recent studies further highlight the expansive role that GPCRs play in promoting autocrine and paracrine influence on cellular transformation, tumor growth, invasion, and metastasis to distant organs (Ram and Iyengar (2001) Oncogene 20(13), 1601-1606).

### SUMMARY

This invention relates to use of GPCR genes as targets for treating cancer.

In one aspect, the invention features a method of determining whether a subject is suffering from or at risk for developing cancer (e.g., colon, liver, gastric, or prostate cancer, or T cell leukemia). For example, the method includes providing a sample (e.g., a colon, liver, gastric, prostate, or blood sample) from a subject and determining the gene expression level of HM74, LGR6, GPR88, or GPR49 in the sample. If the gene expression level of HM74, LGR6, GPR88, or GPR49 in the sample is higher than that in a sample from a normal subject, it indicates that the subject is suffering from or at risk for developing cancer. The gene expression level of HM74, LGR6, GPR88, or GPR49 can be determined by measuring the amount of the mRNA or the protein of HM74, LGR6, GPR88, or GPR49. The mRNA level can be determined, e.g., by in situ hybridization, PCR, or Northern blot analysis. The protein level can be determined, e.g., by Western blot analysis. In another example, the method includes providing a sample from a subject and determining the protein activity level of HM74, LGR6, GPR88, or GPR49 in the sample. If the protein activity level of HM74, LGR6, GPR88, or GPR49 in the sample is higher than that in a sample from a normal subject, it indicates that the subject is suffering from or at risk for developing cancer. The protein activity level of HM74, LGR6, GPR88, or GPR49 can be determined, e.g., by measuring GDP-GTP exchange on G-protein subunits following activation of HM74, LGR6, GPR88, or GPR49.

In another aspect, the invention features a method of identifying a compound for treating cancer. The method includes contacting a compound with a system (a cell system or a cell-free system) containing an HM74, LGR6, GPR88, or GPR49 gene or an HM74, LGR6, GPR88, or GPR49 gene product, and determining the level of HM74, LGR6, GPR88, or GPR49 gene expression or protein activity in the system. The level of HM74, LGR6, GPR88, or GPR49 gene expression or protein activity in the presence of the compound, if lower than that in the absence of the compound, indicates that the compound is a candidate for treating cancer. Such a compound can be any molecule, e.g., an anti-sense RNA, an antibody or its variant, or a non-peptidyl molecule.

Also within the scope of the invention is a method of treating cancer. The method includes identifying a subject suffering from or being at risk for developing cancer and administering to the subject a composition to decrease the level of HM74, LGR6, GPR88, or GPR49 gene expression or protein activity in the subject.

The invention further features a pharmaceutical composition containing a pharmaceutically acceptable carrier and an effective amount of a compound. The compound, when administered to a subject in need thereof, decreases the level of HM74, LGR6, GPR88, or GPR49 gene expression or protein activity in the subject. Thus, the pharmaceutical composition of the invention can be used for treating cancer.

Moreover, the invention features a packaged product including a container, an effective amount of a compound that decreases the level of HM74, LGR6, GPR88, or GPR49 gene expression or protein activity in a subject, and a legend associated with the container and indicating administration of the compound for treating cancer.

The details of one or more embodiments of the invention are set forth in the accompanying description below. Other features, objects, and advantages of the invention will be apparent from the detailed description, and from the claims.

### DETAILED DESCRIPTION

This invention is based on the unexpected discovery that some GPCR genes are up-regulated in cancer cells. Accordingly, the invention provides methods for diagnosing and treating cancer by targeting these GPCR genes.

A diagnostic method of the invention involves comparing the gene expression or protein activity level of HM74, LGR6, GPR88, or GPR49 in a sample prepared from a subject with that in a sample prepared from a normal subject, i.e., a subject who does not suffer from cancer. A higher gene expression or protein activity level of HM74, LGR6, GPR88, or GPR49 indicates that the subject is suffering from or at risk for developing cancer. For example, if the gene expression level in a test subject is 3-fold higher than that in a normal subject as determined by the method described in the examples below or any analogous methods, the test subject is identified as being suffering from or at risk for developing cancer. The method of the invention can be used on its own or in conjunction with other procedures to diagnose cancer.

The gene expression level of HM74, LGR6, GPR88, or GPR49 can be determined at either the mRNA level or the protein level. Methods of measuring mRNA levels in a tissue sample are known in the art. In order to measure mRNA levels, cells can be lysed and the levels of mRNA in the lysates or in RNA purified or semi-purified from the lysates can be determined by any of a variety of methods including, without limitation, hybridization assays using detectably labeled gene-specific DNA or RNA probes and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

Methods of measuring protein levels in a tissue sample are also known in the art. Many such methods employ antibodies (e.g., monoclonal or polyclonal antibodies) that bind specifically to the target protein. In such assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to lysates of cells, and others (e.g., immunohistological methods or fluorescence flow cytometry) applied to histological sections or unlysed cell suspensions. Methods of measuring the amount of label depend on the nature of the label and are well known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

The protein activity level of HM74, LGR6, GPR88, or GPR49 can be determined, e.g., by measuring GDP-GTP exchange on G-protein subunits following activation of HM74, LGR6, GPR88, or GPR49. See, e.g., Peltonen et al. (1998) Eur J Pharmacol 355, 275.

The invention also provides a method for identifying and manufacturing compounds (e.g., proteins, peptides, peptidomimetics, peptoids, antibodies, or small molecules) that decrease the gene expression or protein activity level of HM74, LGR6, GPR88, or GPR49 in a system. Compounds thus identified can be used, e.g., for treating cancer.

The candidate compounds can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. (1994) J Med Chem 37, 2678-2685; and Lam (1997) Anticancer Drug Des 12, 145.

Examples of methods for the synthesis of molecular libraries can be found in the art, for example, in: DeWitt et al. (1993) PNAS USA 90, 6909; Erb et al. (1994) PNAS USA 91, 11422; Zuckermann et al. (1994) J Med Chem 37, 2678; Cho et al. (1993) Science 261, 1303; Carrell et al. (1994) Angew Chem Int Ed Engl 33, 2059; Carell et al. (1994) Angew Chem Int Ed Engl 33, 2061; and Gallop et al. (1994) J Med Chem 37, 1233. Methods of making monoclonal and polyclonal antibodies and fragments thereof are also known in the art. See, for example, Harlow and Lane, (1988) Antibodies: A Laboratory Manual, Cold Spring Harbor Laboratory, New York. The term “antibody” includes intact molecules and fragments thereof, such as Fab, F(ab′)₂, and Fv which are capable of binding to an epitopic determinant present in the HM74, LGR6, GPR88, or GPR49 protein.

Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (U.S. Pat. No. 5,223,409), spores (U.S. Pat. No. 5,223,409), plasmids (Cull et al. (1992) PNAS USA 89, 1865-1869), or phages (Scott and Smith (1990) Science 249, 386-390; Devlin (1990) Science 249, 404-406; Cwirla et al. (1990) PNAS USA 87, 6378-6382; Felici (1991) J Mol Biol 222, 301-310; and U.S. Pat. No. 5,223,409).

To identify compounds that decrease the gene expression or protein activity level of HM74, LGR6, GPR88, or GPR49 in a subject, a system containing the HM74, LGR6, GPR88, or GPR49 gene or an HM74, LGR6, GPR88, or GPR49 gene product (mRNA or protein) is contacted with a candidate compound, and the gene expression or protein activity level of HM74, LGR6, GPR88, or GPR49 is evaluated relative to that in the absence of the candidate compound. In a cell system, the cell (e.g., a cancer cell) can be a cell that naturally expresses the HM74, LGR6, GPR88, or GPR49 gene, or a cell that is modified to express a recombinant HM74, LGR6, GPR88, or GPR49 gene, for example, by having the HM74, LGR6, GPR88, or GPR49 gene fused to a heterologous promoter or by having the HM74, LGR6, GPR88, or GPR49 promoter fused to a heterologous gene. The gene expression or protein activity level of HM74, LGR6, GPR88, or GPR49 can be determined according to the methods described in the examples below, or any other methods well known in the art. If the gene expression or protein activity level of HM74, LGR6, GPR88, or GPR49 is lower in the presence of the candidate compound than that in the absence of the candidate compound, the candidate compound is identified as being useful for treating cancer.

This invention further provides a method for treating cancer. Subjects to be treated can be identified, for example, by determining the gene expression or protein activity level of HM74, LGR6, GPR88, or GPR49 in a sample prepared from a subject by methods described above. If the gene expression or protein activity level of HM74, LGR6, GPR88, or GPR49 is higher in the sample from the subject than that in a sample from a normal subject, the subject is a candidate for treatment with an effective amount of compound that decreases the gene expression or protein activity level of HM74, LGR6, GPR88, or GPR49 in the subject. This method can be performed alone or in conjunction with other drugs or therapy.

The term “treating” is defined as administration of a composition to a subject, who has cancer, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject.

In one in vivo approach, a therapeutic composition (e.g., a composition containing a compound identified as described above) is administered to the subject. Generally, the compound is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. For treatment of cancer, the compound can be delivered directly to the cancer tissue.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a polynucleotide, such as one containing a nucleic acid sequence encoding an anti-sense HM74, LGR6, GPR88, or GPR49 RNA, can be delivered to the subject, for example, by the use of polymeric, biodegradable microparticle or microcapsule delivery devices known in the art. Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al. (1995) J Mol Med 73, 479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

The above-described polynucleotide can be an RNA interference agent, i.e., a duplex-containing RNA or a DNA sequence encoding it, which inhibits the expression of HM74, LGR6, GPR88, or GPR4 via RNA interference. RNA interference (RNAi) is a process in which double-stranded RNA (dsRNA) directs homologous sequence-specific degradation of messenger RNA. In mammalian cells, RNAi can be triggered by 21-nucleotide duplexes of small interfering RNA (siRNA) without activating the host interferon response. As RNAi represses the expression of a specific gene, it can be used to treat a disease caused by abnormally high levels of expression of the gene. A duplex-containing RNA can be synthesized by techniques well known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In the above-mentioned polynucleotides (e.g., expression vectors), the nucleic acid sequence encoding an RNAi agent or an anti-sense HM74, LGR6, GPR88, or GPR49 RNA is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. As is well known in the medical art, the dosage for any one subject depends upon many factors, including the subject's weight, body surface area, age, the particular compound to be administered, sex, time and route of administration, general health, and other drugs being administered concurrently. Dosages will vary, but a preferred dosage for administration of polynucleotide is about 10⁶ to 10¹² copies of the polynucleotide molecule. This dose can be repeatedly administered as needed. Routes of administration can be any of those listed above.

Also within the scope of the invention is a pharmaceutical composition that contains a pharmaceutically acceptable carrier and an effective amount of a compound that decreases the gene expression or protein activity level of HM74, LGR6, GPR88, or GPR49 in a subject. The pharmaceutical composition can be used to treat cancer. The pharmaceutically acceptable carrier includes a solvent, a dispersion medium, a coating, an antibacterial and antifungal agent, and an isotonic and absorption delaying agent. The compound can also be packaged in a container with a label or an insert to indicate the intended uses of the compound, i.e., treatment of cancer.

The compound of the invention can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the ligand with a solid carrier and a lubricant. Examples of solid carriers include starch and sugar bentonite.

The compound can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of a composition of the invention can be evaluated both in vitro and in vivo. For example, the composition can be tested for its ability to decrease the level of HM74, LGR6, GPR88, or GPR49 gene expression or protein activity in vitro. For in vivo studies, the composition can be injected into an animal (e.g., an animal model) and its effects on cancer are then accessed. Based on the results, an appropriate dosage range and administration route can be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Profiling of Liver Tissues

Hepatoma Tumor Tissues

Primary HCCs and corresponding noncancerous liver tissues were obtained with informed consent from 40 patients who underwent hepatectomy. Patient profiles were obtained from medical records. Histopathological classification was performed according to the Edmondson grading system; clinical stages were determined according to the Union International Control Cancer TNM Classification. Histological analysis of paraffin embedded tissue was performed to verify the diagnoses. Tumor samples that were completely surrounded by malignant tissue were used in this study.

RNA Extraction and cDNA Preparation

RNA was extracted using an RNeasy kit (Qiagen, Valencia, Calif.) according to manufacturer's instructions. RNA concentration was determined by spectrophotometry and adjusted to a concentration of 200 ng/μl. RNA (2 μg) was reverse-transcribed using Superscript II enzyme (GIBCO BRL, Gaithersburg, Md.) and 0.5 μg oligo(dT)₁₂₋₁₆ (Amersham, Piscataway, N.J.). The reaction mixture was incubated at 42° C. for 50 min, followed by incubation at 72° C. for 15 min. To ensure the fidelity of mRNA extraction and reverse transcription, all samples were subjected to PCR amplification with oligonucleotide primers specific for the constitutively expressed gene GAPDH and normalization.

Quantitative RT-PCR

The mRNA from each tissue sample was subjected to quantitative RT-PCR using 140 primer pairs specifically designed for 140 non-olfactory GPCRs. Quantitative RT-PCR was performed on the LightCycler instrument using SYBR Green I dye. For each sample, the expression level of target GPCRs and the housekeeping gene (GAPDH) were determined. The ratio of GPCRs-to-GAPDH was calculated as the normalized value. All PCR reactions were performed using the LightCycler-FastStart DNA Master SYBR Green I kit (Roche). Cycling conditions were as follows: initial denaturation at 95° C. for 10 min, followed by 40 cycles of 94° C. for 5 sec, 57° C. for 5 sec, and 72° C. for 15 sec. Amplified cDNAs were separated on 1% agarose gels, and the bands were visualized by ethidium bromide staining.

TABLE 1
Primer sequences used for Quantitative RT-PCR

| | | |
|---|---|---|
| GPR49 | Forward primer: | 5′-CACTGTCATTGCGAGC-3′ |
| | Reverse primer: | 5′-CGCAGGGATTGAAGGC-3′ |
| GPR88 | Forward primer: | 5′-CTGTACTGTAATGGTTGCT-3′ |
| | Reverse primer: | 5′-GTCTAACGGGTATCGCTT-3′ |
| HM74 | Forward primer: | 5′-ATAATAACCGCAGCACG-3′ |
| | Reverse primer: | 5′-AACCTTAGGCCGAGTC-3′ |
| LGR6 | Forward primer: | 5′-GACCATCACCAACGGG-3′ |
| | Reverse primer: | 5′-CATGAGTCACACGGGA-3′ |
| GAPDH | Forward primer: | 5′-TGAGCTGAACGGGAAG-3′ |
| | Reverse primer: | 5′-GTGTCGCTGTTGAAGT-3′ |

Results

Of the 140 GPCRs studied, 2 GPCRs (GPR49 and GPR88) were found to be up-regulated in the hepatoma cancer cells. The expression level of GPR49 showed at least 4- to 100-fold increase in approximately 38% of sample pairs. GPR49 was originally isolated as an orphan G protein-coupled receptor with leucine-rich repeat motifs in the N-terminal region (Hsu et al. (1998) Mol Endocrinol 12(12), 1830-1845). Although the endogenous ligand as well as the biological functions of GPR49 has not yet been elucidated, overexpression of GPR49 mRNA was observed in 47% of hepatocellular carcinomas compared with corresponding noncancerous livers (Yamamoto et al. (2003) Hepatology 37(3), 528-533). GPR88 was originally cloned as a striatum-specific orphan GPCR with highest level of sequence homology to receptors for biogenic amines (Mizushima et al. (2000) Genomics 69(3), 314-321). The expression pattern of GPR88 in human as well as in rodent was restricted in the striatum of brain tissue. It was found that some hepatocellular carcinoma samples showed marked up-regulation of GPR88. In contrast, noncancerous livers showed only low levels of GPR88. The average expression level in hepatocellular carcinoma was 18-fold higher than that in noncancerous liver. Overexpression (tumor/normal ratio >3) was found in 15 of 40 hepatocellular carcinomas (38%). The primer sequences of GPR49 and GPR88 used in quantitative RT-PCR reaction are listed in Table 1 above.

Profiling of Colon Cancer Tissues

Colon Tumor Tissue

Fresh colorectal tissue samples were obtained from the cancerous and noncancerous parts of surgical specimens. Immediately after surgical removal, tissues were grossly dissected by a pathologist, snap frozen and stored in liquid nitrogen until analysis. Histological analysis of paraffin embedded tissue was performed to verify the diagnoses. Tumor samples that were completely surrounded by malignant tissue were used in this study. For in-situ hybridization studies, all samples were immediately frozen and embedded in TissueTek OCT medium (Sakura, Tokyo, Japan) and stored at −80° C. until further analysis.

Results

Of the 140 GPCRs investigated, 3 GPCRs (GPR49, HM74 and LGR6) were found to be up-regulated in the colon cancer tissue. Mean expression of GPR49 was 13-fold higher in the cancerous parts of the colon cancers. Overexpression (tumor/normal ratio >3) was found in 32 of 40 colon cancers (80%). The expression level of HM74 was found to be elevated in 30% tissue pairs. Furthermore, elevated expression of LGR6 was also found in 15 of 40 colon cancer samples (38%). The primer sequences of GPR49, HM74, and LGR6 are listed in Table 1 above.

TABLE 2
Up-regulated expression of GPCRs in hepatoma or colon cancer

| | | Regulation Status |
|---|---|---|
| | GenBank | (Nos. of positive sample/total |
| Gene name | Accession Number | sample) |
| GPR49 | NM_003667 | Up in hepatoma (15/40) |
| | | Up in colon cancer (32/40) |
| GPR88 | NM_022049 | Up in hepatoma (15/40) |
| HM74 | NM_006018 | Up in colon cancer (12/40) |
| LGR6 | AK027377 | Up in colon cancer (15/40) |

Expression of GPCRS in Tumor Cell Lines
Cell Line Information

To determine whether the elevated expression of GPCRs can be detected in cell lines of various origins, the expression levels of GPR49, GPR88, HM74, and LGR6 were examined in 23 human tumor cell lines. Cells were grown to 90% confluency, and total RNA was prepared using the RNeasy kit (Qiagen, Valencia, Calif.) according to the manufacturer's instructions. Gene expression level was determined by quantitative RT-PCR using the primers listed in Table 1 above.

TABLE 3
Cell lines used for GPCR profiling

| Cell name | Cell origin |
|---|---|
| MDA-MB-231 | Human breast adenocarcinoma |
| MDA-MB-435 | Human breast adenocarcinoma |
| MCF-7 | Human breast carcinoma |
| DU4475 | Human breast carcinoma, metastatic cutaneous nodule |
| DLD-1 | Human colon adenocarcinoma |
| LoVo | Human colon adenocarcinoma |
| LS174T | Human colon adenocarcinoma |
| HT-29 | Human colon adenocarcinoma |
| T-84 | Human colon adenocarcinoma |
| SW403 | Human colon adenocarcinoma |
| SW480 | Human colon adenocarcinoma |
| WiDr | Human colon adenocarcinoma |
| AGS | Human gastric adenocarcinoma |
| NUGC | Human gastric cancer cell |
| HepG2 | Human hepatocellular carcinoma |
| Huh-7 | Human liver cancer |
| HH | Human cutaneous T cell leukemia/lymphoma |
| MOLT4 | Human peripheral blood, acute T lymphoblastic leukemia |
| Jurkat J45.01 | Human T lymphocyte, acute T cell leukemia |
| PC-3 | Human prostate adenocarcinoma |
| DU145 | Human prostate carcinoma |
| 22RV1 | Human prostate carcinoma |
| LNCaP | Human prostate carcinoma |

Results

The expression levels of GPCRs in human cancer cell lines were normalized to the levels of GAPDH in individual samples. It was found that GPR49 was expressed in high abundance in human hepatoma cell line HepG2 and Huh7 as well as in human colon cancer cell line LoVo. Furthermore, gastric cancer AGS cells expressed highest level of GPR49. These results further confirmed the potential roles of GPR49 in tumor malignancy. In contrast, none of the breast cancer cell lines in this study (including MCF-7, MDA-MB-231, and MDA-MB-435) expressed significant levels of GPR49. HM74 was significantly expressed in several cancer cells, including hepatoma cells HepG2 and Huh7, colon cancer cells WiDr, SW403, and HT-29, gastric cancer cells NUGC, prostate cancer cells 22RV1, and HH T cell leukemia. In contrast, expression of GPR88 was only found in HH cells. Expression of LGR6 was more restricted to colon cancer cell lines, including SW403, SW480, WiDr, T-84, LoVo, and DLD-1.

Expression of GPR49 in Colon Cancer Tissue

The biopsy samples used to study gene expression in hepatoma and colon cancer contained mixed populations of normal and cancer cells. Therefore, in situ hybridization was used to examine the cellular localization of the GPCR of interest.

Tissue Sections

Tumor samples were obtained from Chung-Gung Memorial Hospital. Tumor tissues were dissected and embedded in OCT (optimal cutting temperature) immediately after surgery. Tissue blocks were stored in −80° C. refrigerator before sectioning. Sequential frozen sections (10 μm) were prepared using Leica CM1900 and thaw-mounted onto gelatin-coated slides. The slides were fixed with 4% paraformaldehyde for 10 minutes followed by 15% sucrose, and then air-dried overnight. The slides were covered with foil and stored at −80° C. until hybridization. Tissue sections were stained with hematoxylin/eosin (H&E) for morphological examination.

Probe Synthesis

DIG-labeled RNA probes were prepared using PCR amplification followed by in vitro transcription. Briefly, the selected regions of the target gene were amplified in PCR reactions, and the amplification products were verified by agarose gel electrophoresis. The DNA was then purified with phenol-chloroform extraction and resuspended in DEPC-treated water for storage at −20° C. RNA probes were then prepared using in vitro transcription, and the labeling efficiency was determined by direct detection. Antisense RNA probes for GPR49 in situ hybridization are: forward primer 5′-GATCAGAATTGGAGTGTGGACCAT-3′ and reverse primer 5′-TGTCGTGCAAAGCTGCCAAAAGTG-3′.

In situ Hybridization

The frozen sections were thawed and washed with 2×SSC. Sections were then digested with proteinase K (1 μg/ml) for 30 minutes at 37° C. followed by acetylation with 0.1 M triethanolamine-HCl. After prehybridization with hybridization buffer for 2 hrs at (Tm-25)° C., sections were hybridized with 50 μl DIG-labeled antisense RNA probe (5 ng/μl) for 18 hrs at the same temperature (hybridization with sense probes were included as controls). Unhybridized single stranded RNA was then digested with RNase A (10 μg/ml RNase A in 10 mM Tris-HCl pH 8.0, 0.5 M NaCl, 1 mM EDTA) at 37° C. for 30 minutes. After stringent washing procedures with SSC, signals were detected with alkaline phosphatase conjugated anti-DIG antibody (Roche, 500-fold dilution in 0.1 M Tris-HCl, 0.15 M NaCl, pH 8.0) and the substrate BCIP-NBT (Sigma). Sections were incubated with anti-DIG antibody at RT for 4 hrs, and signals developed in BCIP-NBT at RT for 45 minutes to 1 hr. After counterstained with 0.2% methylgreen, the sections were air-dried and mounted with Glycer-gel mounting media (Dako). The signals were examined under microscopy (Olympus BX 40) and recorded using digital camera (Olympus C-4040).

Results

It was found that the mRNA level of GPR49 was markedly higher in most of the colon cancers. In order to further confirm these findings, additional studies were performed to determine the histological distribution GPR49 mRNA in specimens from cancerous parts of colon cancer and the corresponding normal colon mucosa using in situ hybridization. High abundance of GPR49 transcript was specifically detected in transformed epithelial cells but not in normal mucosa cells. In order to demonstrate the specificity of the probe, specimens derived from cancerous parts were hybridized with sense and antisense probes of GPR49. Only the antisense probe produced strong signal in cancer specimens. The preferential localization of GPR49 in cancer cells suggests that GPR49 is a useful diagnostic marker as well as a potential therapeutic target.

Further, GPR49 was stably expressed in a human colon cancer cell line (SW480), followed by growth experiments both in vitro and in vivo. It was found that increased GPR49 expression promoted tumor growth, indicating that GPR49 can be used as a diagnostic marker and a therapeutic target of cancer.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of determining whether a subject is suffering from or at risk for developing cancer, the method comprising:
providing a sample from a subject; and
determining a level of HM74 or LGR6 gene expression or protein activity in the sample,
wherein the level of HM74 or LGR6 gene expression or protein activity in the sample, if higher than that in a sample from a normal subject, indicates that the subject is suffering from or at risk for developing cancer.

2. The method of claim 1, wherein the cancer is colon, liver, gastric, or prostate cancer, or T cell leukemia.

3. A method of determining whether a subject is suffering from or at risk for developing cancer, the method comprising:
providing a sample from a subject; and
determining a level of GPR88 gene expression or protein activity in the sample, wherein the level of GPR88 gene expression or protein activity in the sample, if higher than that in a sample from a normal subject, indicates that the subject is suffering from or at risk for developing cancer.

4. The method of claim 3, wherein the cancer is liver cancer or T cell leukemia.

5. A method of determining whether a subject is suffering from or at risk for developing colon or gastric cancer, the method comprising:
providing a sample from a subject; and
determining a level of GPR49 gene expression or protein activity in the sample, wherein the level of GPR49 gene expression or protein activity in the sample, if higher than that in a sample from a normal subject, indicates that the subject is suffering from or at risk for developing colon or gastric cancer.

6. A method of identifying a compound for treating cancer, the method comprising:
contacting a compound with a system containing an HM74 or LGR6 gene or an HM74 or LGR6 gene product; and
determining a level of HM74 or LGR6 gene expression or protein activity in the system,
wherein the level of HM74 or LGR6 gene expression or protein activity in the presence of the compound, if lower than that in the absence of the compound, indicates that the compound is a candidate for treating cancer.

7. The method of claim 6, wherein the cancer is colon, liver, gastric, or prostate cancer, or T cell leukemia.

8. A method of identifying a compound for treating cancer, the method comprising:
contacting a compound with a system containing a GPR88 gene or a GPR88 gene product; and
determining a level of GPR88 gene expression or protein activity in the system, wherein the level of GPR88 gene expression or protein activity in the presence of the compound, if lower than that in the absence of the compound, indicates that the compound is a candidate for treating cancer.

9. The method of claim 8, wherein the cancer is liver cancer or T cell leukemia.

10. A method of identifying a compound for treating colon or gastric cancer, the method comprising:
contacting a compound with a system containing a GPR49 gene or a GPR49 gene product; and
determining a level of GPR49 gene expression or protein activity in the system, wherein the level of GPR49 gene expression or protein activity in the presence of the compound, if lower than that in the absence of the compound, indicates that the compound is a candidate for treating colon or gastric cancer.

11. The method of claim 10, wherein the compound is an antibody.

12. The method of claim 11, wherein the antibody is a monoclonal antibody.

13. A method of treating cancer, the method comprising:
identifying a subject suffering from or being at risk for developing cancer; and
administering to the subject a composition to decrease a level of HM74 or LGR6 gene expression or protein activity in the subject.

14. The method of claim 13, wherein the cancer is colon, liver, gastric, or prostate cancer, or T cell leukemia.

15. A method of treating cancer, the method comprising:
identifying a subject suffering from or being at risk for developing cancer; and
administering to the subject a composition to decrease a level of GPR88 gene expression or protein activity in the subject.

16. The method of claim 15, wherein the cancer is liver cancer or T cell leukemia.

17. A method of treating colon or gastric cancer, the method comprising:
identifying a subject suffering from or being at risk for developing colon or gastric cancer; and
administering to the subject a composition to decrease a level of GPR49 gene expression or protein activity in the subject.

18. A pharmaceutical composition comprising a pharmaceutically acceptable carrier and an effective amount of a compound that decreases a level of HM74 or LGR6 gene expression or protein activity in a subject.

19. The composition of claim 18, wherein the subject suffers from or is at risk for developing cancer.

20. The composition of claim 19, wherein the subject suffers from or is at risk for developing colon, liver, gastric, or prostate cancer, or T cell leukemia.

21. A pharmaceutical composition comprising a pharmaceutically acceptable carrier and an effective amount of a compound that decreases a level of GPR88 gene expression or protein activity in a subject.

22. The composition of claim 21, wherein the subject suffers from or is at risk for developing cancer.

23. The composition of claim 22, wherein the subject suffers from or is at risk for developing liver cancer or T cell leukemia.

24. A packaged product comprising:
a container;
an effective amount of a compound that decreases a level of GPR49 gene expression or protein activity in a subject; and
a legend associated with the container and indicating administration of the compound for treating colon or gastric cancer.


SEQUENCE LISTING (12 sequences)

SEQ ID NO: 1 - DNA, 16 bp, Artificial Sequence
  Primer
cactgtcattgcgagc

SEQ ID NO: 2 - DNA, 16 bp, Artificial Sequence
  Primer
cgcagggattgaaggc

SEQ ID NO: 3 - DNA, 19 bp, Artificial Sequence
  Primer
ctgtactgtaatggttgct

SEQ ID NO: 4 - DNA, 18 bp, Artificial Sequence
  Primer
gtctaacgggtatcgctt

SEQ ID NO: 5 - DNA, 17 bp, Artificial Sequence
  Primer
ataataaccgcagcacg

SEQ ID NO: 6 - DNA, 16 bp, Artificial Sequence
  Primer
aaccttaggccgagtc

SEQ ID NO: 7 - DNA, 16 bp, Artificial Sequence
  Primer
gaccatcaccaacggg

SEQ ID NO: 8 - DNA, 16 bp, Artificial Sequence
  Primer
catgagtcacacggga

SEQ ID NO: 9 - DNA, 16 bp, Artificial Sequence
  Primer
tgagctgaacgggaag

SEQ ID NO: 10 - DNA, 16 bp, Artificial Sequence
  Primer
gtgtcgctgttgaagt

SEQ ID NO: 11 - DNA, 24 bp, Artificial Sequence
  Primer
gatcagaattggagtgtggaccat

SEQ ID NO: 12 - DNA, 24 bp, Artificial Sequence
  Primer
tgtcgtgcaaagctgccaaaagtg